(19) United States
(12) Patent Application Publication
(10) Pub. No.: US 2025/0057586 A1
(43) Pub. Date: Feb. 20, 2025

(54) SURGICAL INSTRUMENT WITH FLUSH PORT

(71) Applicant: RIVERFIELD INC., Tokyo (JP)

(72) Inventors: Koki Shindo, Tokyo (JP); Kyohei Takikawa, Tokyo (JP)

(73) Assignee: RIVERFIELD INC., Tokyo (JP)

(21) Appl. No.: 18/936,272

(22) Filed: Nov. 4, 2024

Related U.S. Application Data
(63) Continuation of application No. PCT/JP2022/020810, filed on May 19, 2022.

(51) Int. Cl. A61B 18/14; A61B 18/00

(52) U.S. Cl. CPC A61B 18/1442; A61B 2018/0091; A61B 2018/1452; A61B 2218/002

(57) ABSTRACT

A surgical instrument includes a housing that defines an inner space, and a shaft that is in fluid communication with the inner space. The housing includes a flush port and an aperture. A center axis of the shaft is interposed between the flush port and the aperture.

### CROSS REFERENCE TO RELATED APPLICATION

This application is a continuation application of International Patent Application No. PCT/JP2022/020810 filed on May 19, 2022, the contents of which being incorporated by reference herein in its entirety.

### BACKGROUND

The present disclosure relates to a surgical instrument.

A surgical instrument may be cleaned and sterilized before or after use.

As a method of cleaning the surgical instrument, a method that uses a cleaning solution is known. For example, a method of cleaning the surgical instrument may be performed by supplying the cleaning solution to an inside of the surgical instrument.

### SUMMARY

It is an aspect to provide a surgical instrument that easily inhibits a deterioration of cleaning efficiency.

According to an aspect of one or more embodiments, there is provided a surgical instrument comprising a housing including an inner space; and a shaft extending from the housing and communicating with the inner space, wherein the housing comprises a flush port that is a through hole for a cleaning solution to enter the inner space of the housing from outside of the housing, and at least one aperture from which the cleaning solution flows to the outside of the housing, and wherein the flush port is situated at a position such that a center axis of the shaft is interposed between the flush port and the at least one aperture.

According to another aspect of one or more embodiments, there is provided a surgical instrument comprising a housing including an inner space; and a shaft extending from the housing and communicating with the inner space, wherein the housing comprises a flush port that is a through hole for a cleaning solution to enter the inner space of the housing from outside of the housing, and wherein the flush port is situated in an area of the housing closer to the shaft than to a center of the housing in a direction in which the shaft extends.

According to yet another aspect of one or more embodiments, there is provided a surgical instrument comprising a housing that defines an inner space; and a shaft that is in fluid communication with the inner space, wherein the housing comprises a flush port and at least one aperture, and wherein a center axis of the shaft is interposed between the flush port and the at least one aperture.

### BRIEF DESCRIPTION OF THE DRAWINGS

FIG. 1 is a front view describing a configuration of a surgical instrument, according to some embodiments.

FIG. 2 is a perspective view describing an outer configuration of a housing and a cover of the surgical instrument of FIG. 1, according to some embodiments.

FIG. 3 is a perspective view describing an inner configuration of the cover of the surgical instrument of FIG. 1, according to some embodiments.

FIG. 4 is a perspective view describing an outer configuration of a base of the surgical instrument of FIG. 1, according to some embodiments.

FIG. 5 is a perspective view describing an inner configuration of the base of the surgical instrument of FIG. 1, according to some embodiments.

FIG. 6 is a cross-sectional view describing a configuration of an inside of a shaft of the surgical instrument of FIG. 1 taken along arrows VI-VI in FIG. 1, according to some embodiments.

FIG. 7 is a schematic diagram describing a flow of a cleaning solution in a surgical instrument, according to some embodiments.

FIG. 8 is a schematic diagram describing a flow of a cleaning solution in a housing, according to some embodiments.

FIG. 9 is a schematic diagram describing a flow of a cleaning solution in a shaft, according to some embodiments.

FIG. 10 is a schematic diagram describing a flow of a different cleaning solution in a surgical instrument, according to some embodiments.

### DETAILED DESCRIPTION

A surgical instrument may include a cartridge having an inner space, a cylindrical shaft extending from the cartridge, and an end effector, such as a forceps, disposed at a leading end of the cylindrical shaft. The surgical instrument may be cleaned and sterilized before or after use.

As a method of cleaning the surgical instrument, a method that uses a cleaning solution may be used. For example, a method of cleaning the surgical instrument may include supplying the cleaning solution in an inside of the cartridge and in an inside of the shaft.

In some cases, the cleaning solution may be supplied through a flush port disposed in the cartridge and a main flush port which is different from the flush port. The cleaning solution supplied through the flush port may be supplied to the inside of the cartridge. The inside of the cartridge may be cleaned with the cleaning solution supplied from the flush port.

The cleaning solution supplied through the main flush port may be supplied to the inside of the shaft via a tube extending from the main flush port. The tube may be situated in the inside of the cartridge, from the main flush port to the inside of the shaft. The inside of the shaft may be cleaned with the cleaning solution supplied through the main flush port and the tube.

Both the cartridge and the shaft may have an opening where they are connected to each other. In other words, an inner space of the cartridge may communicate with an inner space of the shaft. Most of the cleaning solution used to clean the inside of the cartridge may be discharged to outside of the cartridge, while a part of the cleaning solution used to clean the inside of the cartridge may flow from the inner space of the cartridge into the inner space of the shaft.

It is often the case that the shaft does not have an opening for the cleaning solution to flow to the outside. In such a configuration, the cleaning solution in the shaft flows to the outside from the shaft via the cartridge. Thus, it has been necessary to bring the cleaning solution that had entered the shaft from the cartridge after cleaning back to the cartridge to discharge the cleaning solution to the outside from the cartridge. Accordingly, the time required for cleaning the surgical instrument has tended to be long, and therefore, a cleaning efficiency has tended to be poor. Hereinafter, the cartridge is also referred to as “housing” in the present disclosure.

It is thus an aspect to provide a surgical instrument that easily inhibits a deterioration of the cleaning efficiency.

A surgical instrument according to some embodiments may include a housing including an inner space, and a shaft formed into a cylindrical shape extending from the housing and communicating with the inner space. The housing may include a flush port that is a through hole for a cleaning solution to enter from the outside, and at least one aperture from which the cleaning solution flows to the outside. The flush port may be situated at a position that allows a center axis of the shaft to be interposed between the flush port and the at least one aperture.

According to this configuration, the cleaning solution that enters the inside of the housing from the flush port crosses the center axis of the shaft and is likely to flow to the outside of the housing from the at least one aperture. The direction in which the cleaning solution flows is a direction that crosses the center axis of the shaft. Among components of the direction of flow of the cleaning solution, a component of the flow towards the inside of the shaft is likely to be smaller than a component of the flow towards the at least one aperture. In other words, the direction of the flow of the cleaning solution is less likely to be directed from the inner space of the housing towards the inside of the shaft.

In some embodiments, the flush port may be situated such that an axis of the flush port extends through a position different from a position where the center axis of the shaft passes through.

Due to such a configuration, a main direction of the flow of the cleaning solution that enters from the flush port along the axis of the flush port is likely to be different from a direction towards the inside of the shaft. Thus, the cleaning solution supplied to the inside of the housing through the flush port is less likely to enter the inside of the shaft from the inner space of the housing. Hereinafter, the positional relationship between the flush port and the shaft, in which the axis of the flush port extends through a position different from a position through which the center axis of the shaft extends, is also referred to as a twisted positional relationship.

In some embodiments, the flush port may be situated in an area of the housing closer to the shaft than the at least one aperture is.

Due to such a configuration, among the components of the direction of the flow of the cleaning solution that entered the inside of the housing from the flush port, a component of the negative direction of the Z-axis (a direction away from the shaft along the center axis of the shaft) is likely to increase. The cleaning solution is less likely to flow from the inner space of the housing to the inside of the shaft.

A surgical instrument according to some embodiments may include a housing including an inner space, and a shaft formed into a cylindrical shape extending from the housing and communicating with the inner space. The housing may include a flush port that is a through hole for a cleaning solution to enter from an outside. The flush port may be situated in an area in the housing closer to the shaft than to the center of the housing in a direction in which the shaft extends.

According to some embodiments, a main direction of flow of the cleaning solution supplied from the flush port to the inside of the housing is likely to be directed to the center of the housing. In other words, the cleaning solution is likely to flow in a direction away from the shaft. The cleaning solution is less likely to flow from the inner space of the housing to the inside of the shaft.

In some embodiments, the flush port may be situated such that the axis of the flush port passes and extends through a position different from the position through which the center axis of the shaft extends.

Due to such a configuration, the main direction of flow of the cleaning solution from the flush port along the axis of the flush port is likely to be different from the direction towards the inside of the shaft. The cleaning solution is less likely to enter the inside of the shaft from the inner space of the housing.

In some embodiments, the housing may include at least one aperture through which the cleaning solution flows to the outside, and the at least one aperture may be situated at a position that allows the center axis of the shaft to be interposed between the at least one aperture and the flush port.

Due to such a configuration, the main direction of flow of the cleaning solution is likely to be directed towards the at least one aperture. Thus, the direction of flow of the cleaning solution is likely to cross the center axis of the shaft. Among the components of the direction of flow of the cleaning solution, a component of flow towards the inside of the shaft is likely to be smaller than components of flow towards the at least one aperture. In other words, the direction of flow of the cleaning solution is less likely to be directed from the inner space of the housing towards the inside of the shaft.

According to the surgical instrument of various embodiments, the flush port may be situated at a position that allows the center axis of the shaft to be interposed between the flush port and the at least one aperture. Thus, the flow of the cleaning solution is less likely to be directed from the inner space of the housing towards the inside of the shaft, which yields an effect that a deterioration of cleaning efficiency is easily inhibited.

According to the surgical instrument of various embodiments, the flush port may be situated in an area of the housing closer to the shaft than to the center of the housing in a direction in which the shaft extends. Thus, the direction of flow of the cleaning solution is less likely to be directed from the inner space of the housing towards the inside of the shaft, which yields an effect that a deterioration of cleaning efficiency is easily inhibited.

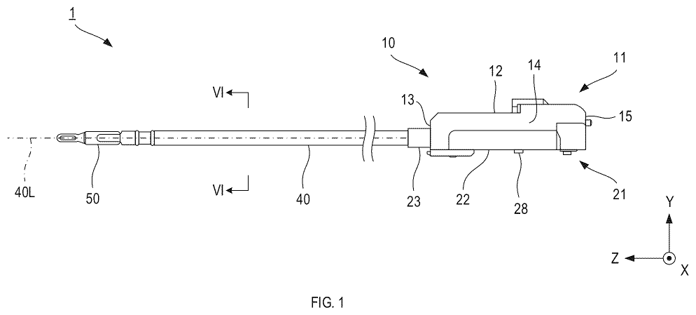

A surgical instrument 1 according to some embodiments will be explained with reference to FIG. 1 to FIG. 8. The surgical instrument 1 is an instrument used in surgeries and attached to a master-slave-type surgical robot. In some embodiments, the surgical instrument 1 may be attached to surgical robots other than the master-slave-type surgical robot. In some embodiments, the surgical instrument 1 may be an instrument that is not attached to a surgical robot. FIG. 1 to FIG. 8 do not illustrate a surgical robot for conciseness and ease of understanding.

In some embodiments, the surgical instrument 1 may be an instrument that is moved by a driving force transmitted from outside. The driving force is generated in an external drive source, such as an actuator, and is transmitted to the surgical instrument 1 via a transmitting body, such as a wire.

As shown in FIG. 1, in some embodiments, the surgical instrument 1 may include a housing 10 and a shaft 40. In some embodiments, the surgical instrument 1 may include an end effector 50.

Hereinafter, explanations will be given by referring to an X-direction, a Y-direction, and a Z-direction which will be explained below. The X-direction and the Y-direction are directions extending along a plane situated perpendicular to a center axis 40L of the shaft 40 that extends along the surface of FIG. 1. The X-direction and the Y-direction are perpendicular to each other. The plane situated perpendicular to the center axis 40L of the shaft 40 is perpendicular to the surface of FIG. 1.

In other words, the X-direction is a direction extending along a mounting surface 22 of a base 21 of the housing 10. The positive direction of the X-direction is a direction that extends outward from the surface of FIG. 1. In other words, the positive direction of the Y-direction is a direction from the base 21 of the housing 10 towards a cover 11. The Z-direction is a direction parallel to the center axis 40L. The positive direction of the Z-direction is a direction from the housing 10 towards the end effector 50.

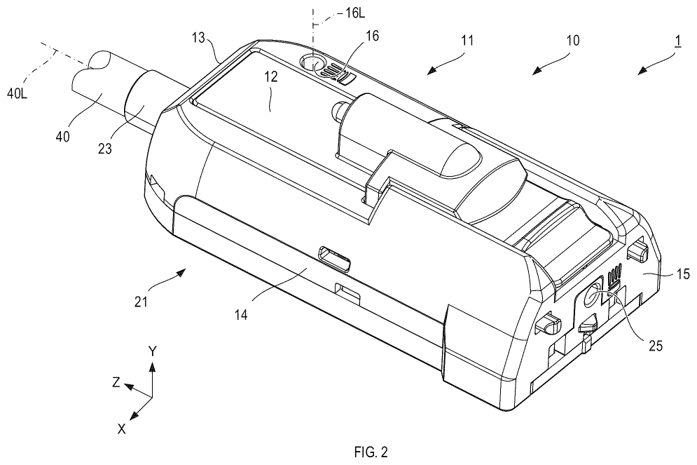

The housing 10 is a container having an inner space. The housing 10 is a part of the surgical instrument 1 that is attached to a surgical robot. As shown in FIG. 2, the housing 10 includes the cover 11 and the base 21.

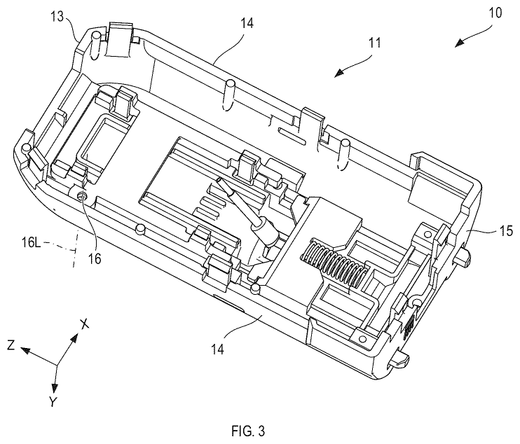

The cover 11 is a member that forms an outer shape and the inner space of the housing 10 together with the base 21. As shown in FIG. 2 and FIG. 3, the cover 11 includes an upper surface 12, a front surface 13, lateral surfaces 14, 14, and a back surface 15.

The upper surface 12 is a surface extending along a plane Z-X and is situated at the positive side in the Y-direction in the cover 11. The front surface 13 is a surface extending along a plane X-Y and is situated at the positive side in the Z-direction in the cover 11. The lateral surfaces 14, 14 are surfaces extending along a plane Y-Z and is situated at the positive side and the negative side in the X-direction in the cover 11. The back surface 15 is a surface extending along a plane X-Y and is situated at the negative side in the Z-direction in the cover 11.

The upper surface 12, the front surface 13, the lateral surfaces 14, 14, and the back surface 15 each may only include a flat surface, each may include a combination of a flat surface and a curved surface, or each may only include a curved surface. The upper surface 12, the front surface 13, the lateral surfaces 14, 14, and the back surface 15 each may also include a protrusion or a recess.

As shown in FIG. 2, the cover 11 includes a flush port 16. In some embodiments, the flush port 16 may be disposed on the upper surface 12 of the cover 11. In some embodiments, the flush port 16 may be disposed on the front surface 13 of the cover 11, or on the lateral surfaces 14, 14.

The flush port 16 is situated at a position that allows the center axis 40L to be interposed between the flush port 16 and the base 21 of the cover 11. More specifically, the flush port 16 is situated at a position that allows the center axis 40L to be interposed between the flush port 16 and an aperture 27 of the base 21 which will be mentioned later.

The flush port 16 is a through-hole that passes through the cover 11. The flush port 16 is a through-hole that is used when supplying a cleaning solution from outside of the housing 10 to the inner space of the housing 10. The flush port 16 passes through the upper surface 12 of the cover 11.

In the cover 11, the flush port 16 is situated on the positive side in the Z-direction with respect to the center of the cover 11. In other words, in the area of the cover 11, the flush port 16 is situated in an area that is closer to the shaft 40 than to the center of the cover 11. More specifically, on the upper surface 12, the flush port 16 is situated in an area that is closer to the shaft 40 than to the center of the upper surface 12 in the Z-direction.

When viewed in the Y-direction, the flush port 16 is situated such that an axis 16L of the flush port 16 extends through a position different from a position where the center axis 40L passes through. In other words, the flush port 16 is situated at a position different from the position through which the center axis 40L extends.

More specifically, when viewed in the Y-direction, the flush port 16 is situated away from the center axis 40L towards the negative side in the X-direction. The flush port 16 may be situated at a position away from the center axis 40L towards the positive side in the X-direction.

The axis 16L is an axis that passes through the center of the flush port 16, which is a through-hole. If the through-hole has a shape of a cylinder or a column, the center axis of such a cylinder or a column is the axis 16L.

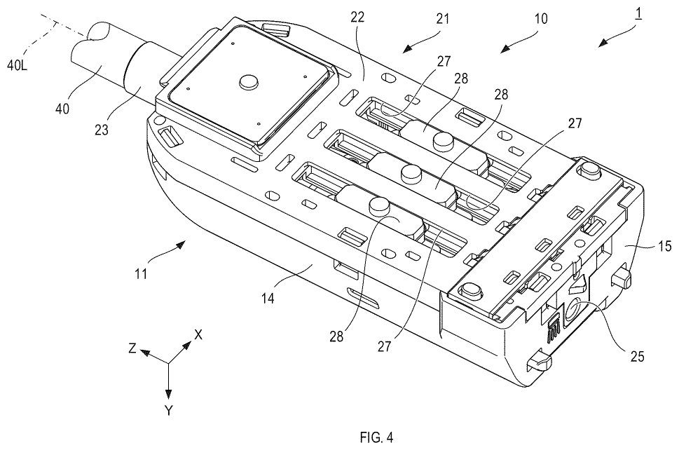

As shown in FIG. 4, the base 21 is a member formed into a shape of a plate that forms an outer shape of the housing 10 along with the cover 11. The base 21 is a portion of the surgical instrument 1 that is attached to the surgical robot. On the base 21, the cover 11 is situated in the positive side in the Y-direction. The surface of the base 21 on the negative side in the Y-direction is the mounting surface 22. The mounting surface 22 is a surface for mounting the surgical instrument 1 on the surgical robot and is a surface that comes in contact with the surgical robot. The surgical robot is not illustrated in FIG. 4.

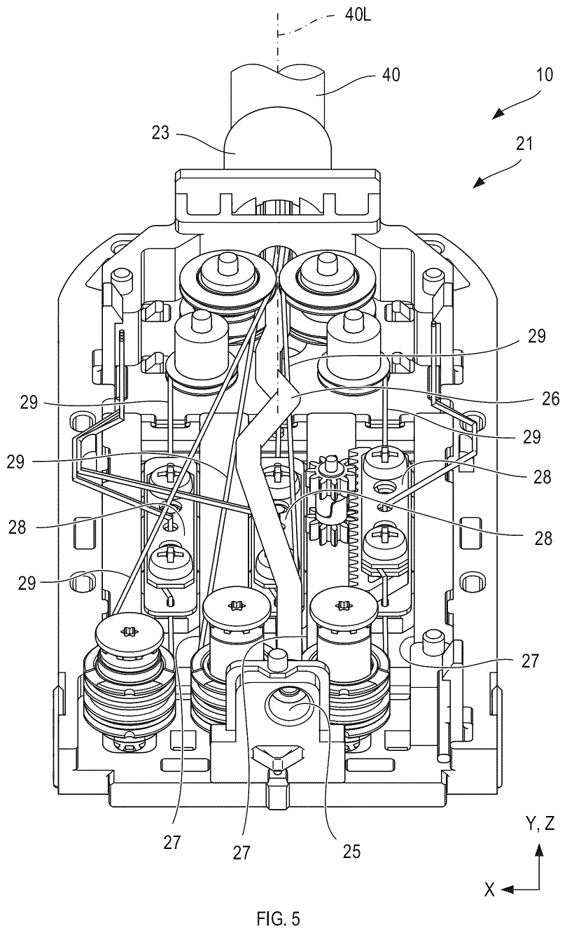

As shown in FIG. 1 and FIG. 5, for example, the base 21 includes a shaft mounting portion 23 for mounting the shaft 40 at one end of the base 21 situated on the positive side in the Z-direction. The shaft mounting portion 23 is disposed at a protrusion of the base 21 on the side of the cover 11 (in other words, on the positive side in the Y-direction). The shaft mounting portion 23 is disposed at the center of the base 21 in the X-direction. The shaft mounting portion 23 has a cylindrical shape protruding towards the positive side in the Z-direction. The shaft 40 is inserted into the shaft mounting portion 23 and retained in an inside of the shaft mounting portion 23.

A main flush port 25 is disposed at one end of the base 21 situated on the negative side in the Z-direction. The main flush port 25 is a through hole that is used to supply the cleaning solution from the outside of the housing 10 to the inside of the shaft 40.

On the other side of the shaft 40 on the base 21, the main flush port 25 is disposed at a portion of the base 21 extending towards the cover 11 side (in other words, on the positive side in the Y-direction). The main flush port 25 is a through hole extending in the Z-direction. In some embodiments, the main flush port 25 may be situated at a position deviated from the center axis 40L of the shaft 40. In some embodiments, the main flush port 25 may be situated at a position situated on the center axis 40L.

A tube 26 is coupled to the main flush port 25 (see FIG. 5). The tube 26 is a member that is used when supplying the cleaning solution to the inside of the shaft 40 along with the main flush port 25.

The tube 26 is a member formed into a cylindrical shape. The tube 26 is a member formed by using a flexible material such as a resin. The tube 26 is situated along the Z-direction. The tube 26 may be situated to have a linear shape, or may be situated to have a shape formed by combining straight lines and curved lines, or may be situated to have a curved shape.

The tube 26 is situated between the flush port 16 and the base 21. More specifically, the tube 26 is situated between the flush port 16 and an aperture 27 of the base 21 which will be explained later.

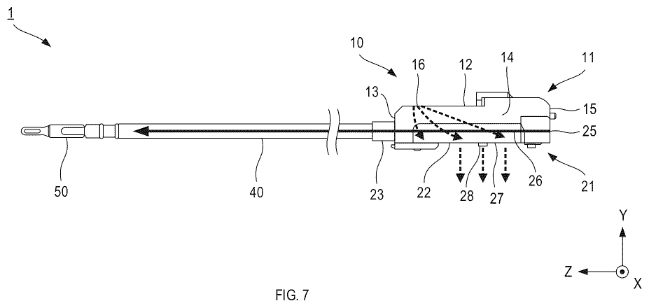

When viewed in the Y-direction, the tube 26 is situated such that the axis 16L of the flush port 16 passes and extends through a position different from a position where the tube 26 passes through (see, e.g., FIG. 7). More specifically, when viewed in the Y-direction, the tube 26 is situated at a position away from the flush port 16 towards the positive side in the X-direction (see, e.g., FIGS. 5 and 7). The tube 26 may be situated at a position away from the flush port 16 towards the negative side in the X-direction.

One end of the tube 26 situated on the negative side in the Z-direction is coupled to the main flush port 25. The other end of the tube 26 situated on the positive side in the Z-direction is situated in the inside of the shaft 40.

As shown in FIG. 4, the base 21 includes three apertures 27, 27, 27 and three sliders 28, 28, 28. Hereinafter, when an explanation is given about a single aperture 27, the other two apertures 27, 27 also have the same configuration. Hereinafter, the aperture 27 is also referred to as a driven groove. Hereinafter, when an explanation is given about a single slider 28, the other two sliders 28, 28 also have the same configuration.

The aperture 27 is a through hole that connects the inner space of the housing 10 to the outside. The aperture 27 is a through hole disposed on the base 21. The aperture 27 is an elongated hole extending in the Z-direction. The three apertures 27, 27, 27 are aligned at equal intervals in the X-direction.

In the Z-direction, the three apertures 27, 27, 27 are situated in a central area of the base 21. The flush port 16 is situated closer to the positive side in the Z-direction than the three apertures 27, 27, 27 are. In other words, the flush port 16 is situated in an area closer to the shaft 40 than the three apertures 27, 27, 27 are.

The number of the aperture 27 can be determined based on movements or the like of a joint and/or a forceps in the end effector 50. In other words, the number of the aperture 27 can be determined based on the movements made in accordance with the specification required in the surgical instrument 1. The number of the aperture 27 may be larger than three or smaller than three depending on the specification required.

The slider 28 is configured to receive a driving force transmitted from the surgical robot and to transmit the driving force to the end effector 50. The slider 28 is attachable to and detachable from the surgical robot.

The slider 28 is situated such that the slider 28 can move relative to the base 21. More specifically, the slider 28 is situated such that the slider 28 can move relative to the base 21 in a straight direction. The slider 28 may be situated such that the slider 28 can rotate relative to the base 21.

The sliders 28, 28, 28 are respectively situated in the three apertures 27, 27, 27. The sliders 28, 28, 28 are situated such that each of the sliders 28, 28, 28 can move in the inside of corresponding one of the apertures 27, 27, 27 in the Z-direction. In other words, the slider 28 is situated such that the slider 28 can move relative to the base 21 in a straight direction. All of the apertures 27, 27, 27 may have one of the sliders 28, 28, 28 situated therein, or only some of the apertures 27, 27, 27 may have one of the sliders 28, 28, 28 situated therein.

As shown in FIG. 5, the base 21 includes three wires 29, 29, 29. The wires 29, 29, 29 are configured to transmit the driving force transmitted to the slider 28 to the end effector 50. One or two wires 29, 29 are situated on the slider 28. Hereinafter, when an explanation is given about a single wire 29, the other two wires 29, 29 also have the same configuration.

The wire 29 is a wire formed into a long shape by using an electrically conductive material. The wire 29 may be formed by using a metallic material used in a manipulator of a surgical robot system, such as stainless steel, tungsten, an alloy containing tungsten, or a piano wire (for example, those specified in JIS G 3522).

As shown in FIG. 1, the shaft 40 is a member formed into a cylindrical shape that extends in the Z-direction from the base 21 of the housing 10. The shaft 40 may have a tubular shape other than the cylindrical shape.

The end effector 50 is situated in one end of the shaft 40 situated on the positive side in the Z-direction. As shown in FIG. 5, the other end of the shaft 40 situated on the negative side in the Z-direction is mounted to the shaft mounting portion 23 of the base 21. The inner space of the cylindrical shaft 40 communicates with the inner space of the housing 10.

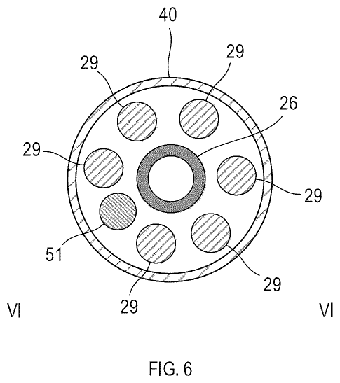

As shown in FIG. 6, the tube 26, the wires 29, and an electrical wiring 51 are situated in the inside of the shaft 40. The electrical wiring 51 is used to supply an electric current to the end effector 50.

The tube 26 is situated near the center in the inside of the shaft 40. The wires 29 and the electrical wiring 51 are situated around the tube 26. More specifically, six wires 29 and one electrical wiring 51 are situated around the tube 26.

The end effector 50 shown in FIG. 1 is an instrument used in a surgery. The end effector 50 has a function of a monopolar electric scalpel and a forceps. The end effector 50 may have a function of only one of the electric scalpel or the forceps, or may also have different functions.

As shown in FIG. 6, the six wires 29 and the electrical wiring 51 situated through the inside of the shaft 40 are coupled to the end effector 50. The end effector 50 is configured such that the six wires 29 open and close the forceps and change the orientation of the forceps. The end effector 50 is configured such that a high frequency current supplied by the electrical wiring 51 is transmitted to the forceps.

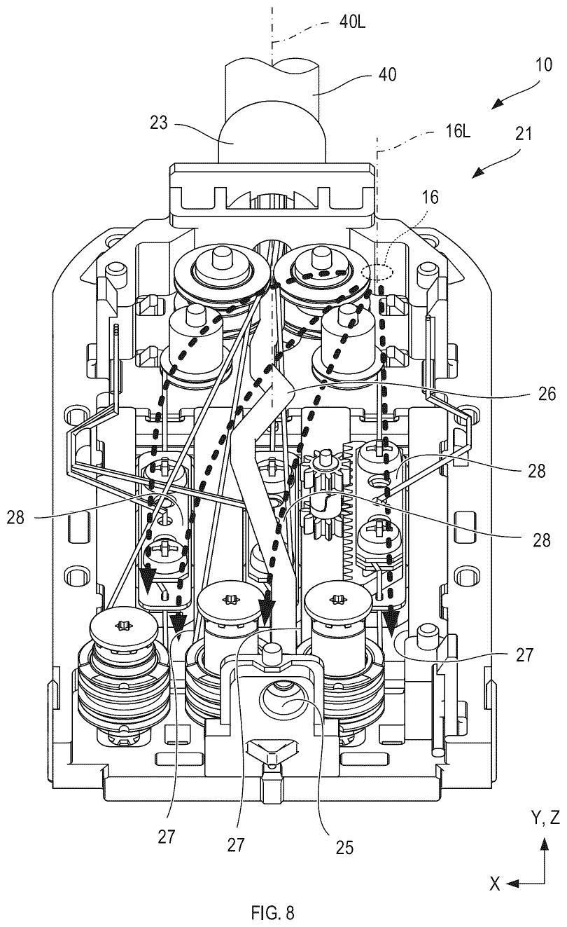

Next, a flow of the cleaning solution when cleaning the surgical instrument 1 configured as mentioned above will be explained with reference to FIG. 7 and FIG. 8. Firstly, a flow of the cleaning solution supplied through the flush port 16 will be explained.

As shown in FIG. 7, the cleaning solution supplied through the flush port 16 flows into the inner space of the housing 10 from the outside of the housing 10. At this time, the cleaning solution flows from the positive side to the negative side in the Y-direction. The cleaning solution spreads and flows in the inner space of the housing 10. The cleaning solution removes contaminants and the like in the inner space of the housing 10. In other words, the cleaning solution cleans the contaminants and the like in the inner space of the housing 10.

The cleaning solution that has flowed into the inner space of the housing 10 then flows through the inside of the housing 10 towards a position where the housing 10 communicates with the outside of the housing 10. More specifically, the cleaning solution flows towards the apertures 27, 27, 27, a connection between the cover 11 and the base 21, and the like.

When comparing the apertures 27, 27, 27 with the connection between the cover 11 and the base 21, a difference is found in the ease of flow of the cleaning solution. In other words, the ease of flow of the cleaning solution from the apertures 27, 27, 27 to the outside of the housing 10 is different from the ease of flow of the cleaning solution from the aforementioned connection to the outside of the housing 10.

A flow passage resistance at a portion of the apertures 27, 27, 27 where the cleaning solution flows is smaller than the flow passage resistance at the connection. Thus, the cleaning solution is likely to flow towards the apertures 27, 27, 27 after spreading in the inner space of the housing 10. The cleaning solution is less likely to flow in other directions.

As shown in FIG. 7 and FIG. 8, the cleaning solution that has flowed into the inner space of the housing 10 flows from the flush port 16 towards the negative side in the Y-direction and is likely to flow towards the negative side in the Z-direction. In other words, the cleaning solution that has flowed into the inner space of the housing 10 is less likely to flow towards the positive side in the Z-direction.

In the Z-direction, the shaft 40 is situated in the positive side with reference to the flush port 16. Thus, the cleaning solution that flowed into the inner space of the housing 10 is less likely to flow towards the shaft 40, and therefore, less likely to flow into the inside of the shaft 40.

A part of the cleaning solution that has flowed into the inner space of the housing 10 is also likely to flow towards the positive side in the X-direction. The shaft 40 extends in the Z-direction. A flow towards the positive side in the X-direction is a flow in a direction crossing the shaft 40, and thus is not a flow towards the inside of the shaft 40. Thus, the cleaning solution that has flowed into the inner space of the housing 10 is less likely to flow towards the inside of the shaft 40, and therefore, less likely to flow into the inside of the shaft 40.

The cleaning solution flows through the flush port 16 into the inner space of the housing 10 without colliding with the tube 26 and without colliding with other elements. Hereinafter, the event that the cleaning solution collides with the tube without colliding with other elements is also referred to as “direct collision”. Accordingly, it is less likely to happen that the flow direction of the cleaning solution is changed to the direction towards the inside of the shaft 40 due to the direct collision with the tube 26.

Next, a flow of the cleaning solution that is supplied from the main flush port 25 will be explained. As shown in FIG. 7 and FIG. 8, the cleaning solution supplied from the main flush port 25 flows into the tube 26. The cleaning solution is guided by the tube 26 to the inside of the shaft 40.

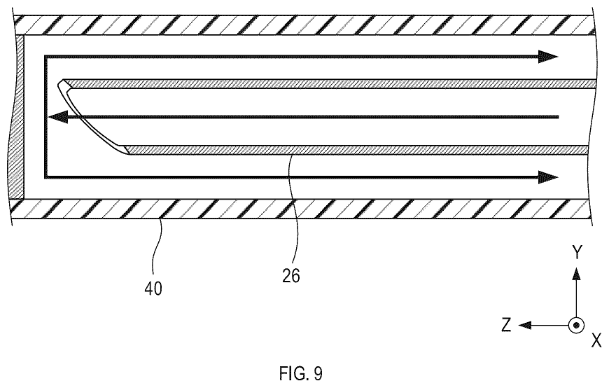

As shown in FIG. 9, the cleaning solution that was guided by the tube 26 flows to the inside of the shaft 40. An end of the shaft 40 on the positive side in the Z-direction is closed. The cleaning solution that flows out from the tube 26 towards the positive direction in the Z-direction therefore changes its direction of flow to the negative direction in the Z-direction in the inside of the shaft 40.

The cleaning solution that changed its direction of flow removes the contaminants and the like that exist in the inside of the shaft 40. In other words, the cleaning solution cleans the inside of the shaft 40. The cleaning solution that returns to the inner space of the housing 10 flows out to the outside of the housing 10 from the apertures 27, 27, 27 and the like.

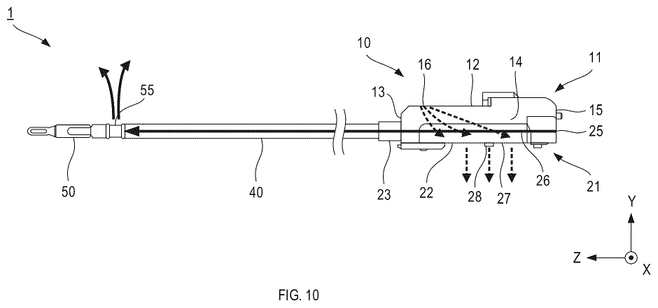

As shown in FIG. 10, in some embodiments, the shaft 40 may include a discharge hole 55 that discharges the cleaning solution to the outside. In FIG. 10, the discharge hole 55 is disposed at a position in the shaft 40 where the end effector 50 is mounted. The discharge hole 55 may be disposed at a position other than where the end effector 50 is mounted.

According to the surgical instrument 1 configured as above, the cleaning solution that flows through the flush port 16 into the inner space of the housing 10 crosses the center axis 40L of the shaft 40 and is likely to flow out from the apertures 27, 27, 27 to the outside of the housing 10. In other words, the main direction of the flow of the cleaning solution is likely to be the direction towards the apertures 27, 27, 27. The direction of the flow of the cleaning solution is likely to be the direction crossing the center axis 40L of the shaft 40.

Among components of the direction of the flow of the cleaning solution, a component of the flow towards the inside of the shaft 40 is likely to be smaller than a component of the flow towards the apertures 27, 27, 27. In other words, the direction of the flow of the cleaning solution is less likely to be directed from the inner space of the housing 10 towards the inside of the shaft 40.

The cleaning solution supplied through the flush port 16 to the inside of the housing 10 is likely to be directed towards the center of the housing 10. In other words, among the components of the direction of the flow of the cleaning solution directed from the flush port 16 towards the apertures 27, 27, 27, components of the negative direction of the Z-axis is likely to increase. For example, the cleaning solution is likely to flow towards the direction away from the shaft 40. Thus, the cleaning solution is less likely to flow from the inner space of the housing 10 to the inside of the shaft 40.

Since the axis 16L of the flush port 16 extends by passing through a position different from a position where the center axis 40L of the shaft 40 passes through, the main direction of the flow of the cleaning solution that enters from the flush port 16 along the axis 16L is likely to be a direction different from the direction towards the inside of the shaft 40. Thus, the cleaning solution is less likely to flow from the inner space of the housing 10 to the inside of the shaft 40.

Accordingly, a need for directing the cleaning solution after cleaning the inner space of the housing 10, which has flowed into the inside of the shaft 40, back from the inside of the shaft 40 to the inner space of the housing 10 to have the cleaning solution flow to the outside of the housing 10 decreases. As a result, the duration of time required for cleaning the surgical instrument 1 is less likely to be prolonged, and thus, a deterioration of cleaning efficiency is likely to be inhibited.

While various embodiments have been described above with reference to the drawings, one of ordinary skill in the art will understand that various changes and modifications may be made thereto in accordance with the concepts set forth in the present disclosure, and all such changes and modifications are to be included within the scope of the appended claims.

1. A surgical instrument comprising:
a housing including an inner space; and
a shaft extending from the housing and communicating with the inner space,
wherein the housing comprises a flush port that is a through hole for a cleaning solution to enter the inner space of the housing from outside of the housing, and at least one aperture from which the cleaning solution flows to the outside of the housing, and
wherein the flush port is situated at a position such that a center axis of the shaft is interposed between the flush port and the at least one aperture.

2. The surgical instrument according to claim 1,
wherein the flush port is situated such that an axis of the flush port passes and extends through a position in the inner space that is different from a position where the center axis of the shaft passes through the inner space.

3. The surgical instrument according to claim 2,
wherein the flush port is situated in an area of the housing closer to the shaft than to the at least one aperture.

4. The surgical instrument according to claim 1,
wherein the flush port is situated in an area of the housing closer to the shaft than to the at least one aperture.

5. The surgical instrument according to claim 1, wherein the shaft is connected to the housing at a proximal end of the shaft, and
wherein the shaft comprises a discharge hole in a distal end of the shaft.

6. The surgical instrument according to claim 1, further comprising a tube,
wherein the housing further comprises a main flush port, and
wherein the tube is in fluid communication with the main flush port and extends through the inner space of the housing and coaxially along an inside of the shaft.

7. A surgical instrument comprising:
a housing including an inner space; and
a shaft extending from the housing and communicating with the inner space,
wherein the housing comprises a flush port that is a through hole for a cleaning solution to enter the inner space of the housing from outside of the housing, and
wherein the flush port is situated in an area of the housing closer to the shaft than to a center of the housing in a direction in which the shaft extends.

8. The surgical instrument according to claim 7,
wherein the flush port is situated such that an axis of the flush port passes and extends through a position in the inner space that is different from a position through which a center axis of the shaft extends through the inner space.

9. The surgical instrument according to claim 7,
wherein the housing includes at least one aperture from which the cleaning solution flows to the outside of the housing, and
wherein the at least one aperture is situated at a position such that a center axis of the shaft is interposed between the at least one aperture and the flush port.

10. The surgical instrument according to claim 7, wherein the shaft is connected to the housing at a proximal end of the shaft, and
wherein the shaft comprises a discharge hole in a distal end of the shaft.

11. The surgical instrument according to claim 7, further comprising a tube,
wherein the housing further comprises a main flush port, and
wherein the tube is in fluid communication with the main flush port and extends through the inner space of the housing and coaxially along an inside of the shaft.

12. A surgical instrument comprising:
a housing that defines an inner space; and
a shaft that is in fluid communication with the inner space,
wherein the housing comprises a flush port and at least one aperture, and
wherein a center axis of the shaft is interposed between the flush port and the at least one aperture.

13. The surgical instrument according to claim 12, wherein the flush port comprises a hole in the housing, and
wherein an axis of the flush port does not intersect the center axis of the shaft.

14. The surgical instrument according to claim 13,
wherein the flush port closer to the shaft than to the at least one aperture in a direction of the center axis of the shaft.

15. The surgical instrument according to claim 12,
wherein the flush port closer to the shaft than to the at least one aperture in a direction of the center axis of the shaft.

16. The surgical instrument according to claim 12, wherein the shaft is connected to the housing at a proximal end of the shaft, and
wherein the shaft comprises a discharge hole in a distal end of the shaft.

17. The surgical instrument according to claim 12, further comprising a tube,
wherein the housing further comprises a main flush port, and
wherein the tube is in fluid communication with the main flush port and extends through the inner space of the housing and coaxially along an inside of the shaft.
